SEQUENCE LISTING (13 sequences)

SEQ ID NO: 1 - DNA, 18 bp, Artificial Sequence
  Description of Artificial Sequence Synthetic primer
gtagtggcttagtccctg

SEQ ID NO: 2 - DNA, 19 bp, Artificial Sequence
  Description of Artificial Sequence Synthetic primer
gagaaagaactggaagagc

SEQ ID NO: 3 - DNA, 19 bp, Artificial Sequence
  Description of Artificial Sequence Synthetic primer
cccatacatatacagccac

SEQ ID NO: 4 - DNA, 19 bp, Artificial Sequence
  Description of Artificial Sequence Synthetic primer
cactccttctctaaaaggg

SEQ ID NO: 5 - DNA, 18 bp, Artificial Sequence
  Description of Artificial Sequence Synthetic primer
gcaaatggttggaactgg

SEQ ID NO: 6 - DNA, 18 bp, Artificial Sequence
  Description of Artificial Sequence Synthetic primer
ctggtacaaccattctgg

SEQ ID NO: 7 - DNA, 17 bp, Artificial Sequence
  Description of Artificial Sequence Synthetic primer
gtaaggctaaggacacc

SEQ ID NO: 8 - DNA, 20 bp, Artificial Sequence
  Description of Artificial Sequence Synthetic primer
gttttgtcaaagaaagggtg

SEQ ID NO: 9 - DNA, 16 bp, Artificial Sequence
  Description of Artificial Sequence Synthetic primer
gtggatcagcaagcag

SEQ ID NO: 10 - DNA, 18 bp, Artificial Sequence
  Description of Artificial Sequence Synthetic primer
gcagaaggagattactgc

SEQ ID NO: 11 - DNA, 16 bp, Artificial Sequence
  Description of Artificial Sequence Synthetic primer
cgcatcctcttcctcc

SEQ ID NO: 12 - DNA, 15 bp, Artificial Sequence
  Description of Artificial Sequence Synthetic primer
caggctgtgttgtcc

SEQ ID NO: 13 - DNA, 18 bp, Artificial Sequence
  Description of Artificial Sequence Synthetic primer
ccatggatgacgatatcg

(19) United States
(12) Patent Application Publication
(10) Pub. No.: US 2015/0285720 A1
(43) Pub. Date: Oct. 8, 2015

(54) METHOD OF FORMALIN BASED TISSUE FIXATION

(71) Applicant: GENERAL ELECTRIC COMPANY, Schenectady, NY (US)

(72) Inventors: Bruce Allan Hay, Niskayuna, NY (US); Brian James Grimmond, Clifton Park, NY (US); Lisa Anne Lowery, Niskayuna, NY (US); Qing Li, Niskayuna, NY (US); Patrick McCoy Spooner, Slingerlands, NY (US); Christine Angela Morton, Troy, NY (US)

(73) Assignee: GENERAL ELECTRIC COMPANY, Schenectady, NY (US)

(21) Appl. No.: 14/247,353

(22) Filed: Apr. 8, 2014

(51) Int. Cl. G01N 1/42; C12Q 1/68; G01N 33/483

(52) U.S. Cl. CPC G01N 1/42; G01N 33/4833; C12Q 1/6806

(57) ABSTRACT

Disclosed are novel cold fixation methods and reagents comprising contacting a biological sample with a reagent for fixation at a temperature of less than 20° C. where the reagent comprises an aqueous buffer, a water soluble alkylnitrile, C2 to C6 alkyl ester, or combination thereof, and formaldehyde to the aqueous buffer solution and removing the biological sample from contact with the reagent.

### SEQUENCE LISTING

The instant application contains a Sequence Listing which has been submitted electronically in ASCII format and is hereby incorporated by reference in its entirety. Said ASCII copy, created on May 8, 2014, is named 272800-1_SL.txt and is 3,282 bytes in size.

### BACKGROUND

Aldeyde fixation, such as formalin fixation is a mainstay of modern histopathologic analysis, yet the procedure has a numerous sources of preanalytical errors related to the processing conditions used. Concerns of workflow and turnaround time drive interest in developing shorter fixation protocols, but rapid protocols can lead to poor histomorphology or inadequate downstream assay results.

More specifically, a role of fixation is for the preservation of antigens and analytes. DNA and RNA analysis of fixed tissue samples is becoming more important due to advances in molecular imaging and genomics. However, DNA for example, is degraded during normal room temperature, 24 hr, formalin fixation, with less DNA degradation with lower temperature fixations. But lower temperature also results in incomplete fixation/poor morphology after 24 h. The obvious need is for a method to speed up cold fixation that maintains the better DNA retention profile but also yields good morphology and allows for completion of the fixation process in a reasonable amount of time (Tokuda et. al., J. Clinical Pathology 1990; 43:748-751).

Additionally, there is a concern that outside of a few clinical applications, tissue fixation is not rigidly standardized in the clinical laboratory. For example, the American Society of Clinical Oncology/College of American Pathologists (ASCO/CAP) guidelines for HER2 IHC call for fixation in neutral buffered formalin for at least 6 hours and no more than 72 hours. While these guidelines are well intentioned, they still allow a 12-fold variation in fixation time and are not meant to represent optimal conditions for all IHC assays.

Methods of fixing tissues samples with strict temperature controls are also described in U.S. patent application Ser. No. 13/372,040 and U.S. patent application Ser. No. 13/302,752. However, there is still a need for improvement due to reduced tissue quality for subsequent histologic and molecular studies.

As such there remains a need for rapid fixation that can be standardized and used robustly for a variety of histopathologic analysis including DNA and RNA analysis.

### BRIEF DESCRIPTION

Disclosed herein are novel cold fixation methods comprising contacting a biological sample with a reagent for fixation at a temperature of less than 20° C. where the reagent comprises an aqueous buffer solution comprising 2-80 volume % of a water soluble alkylnitrile, C₂ to C₆ alkyl ester, or combination thereof, and 0.5 to 20% w/v formaldehyde to the aqueous buffer solution and removing the biological sample from contact with the reagent.

In some embodiments, the method further comprises washing the biological sample with a rinsing liquid to remove excess reagent and processing the sample further for DNA analysis or amplification, RNA analysis or amplification, protein analysis, antigen retrieval, H&E (Hematoxylin and Eosin), immunofluorescence staining (IF), immunohistochemical staining (IHC), fluorescent in-situ hybridization (FISH) or other histological and morphological staining techniques.

Also disclosed are reagents for the cold fixation comprising an aqueous buffer solution comprising 2-80 volume % of a water soluble alkylnitrile, C₂ to C₆ alkyl ester, or combination thereof, and 0.5 to 20% w/v formaldehyde to the aqueous buffer solution.

### DESCRIPTION OF THE FIGURES

FIG. 1 is a schematic representation of a method of fixation using a cold fixation reagent.

FIG. 2 are micrographs of tissue obtained using a 20× objective lens comparing H&E staining for standard room temperature formalin fixation for rat liver, colon, and muscle (No. 1-3, respectively) and low temperature aqueous formalin fixation (No. 4-6 respectively) and, in an embodiment of this invention, low temperature fixation with added acetonitrile (No. 7-9 respectively)

FIG. 3 are micrographs of H&E stained tissue obtained using a 20× objective lens for conditions listed in Table 4 comparing variations in reagent composition, temperature, and duration of fixation.

FIG. 4 is a graphical representation of DNA amplification under various conditions.

FIG. 5 are gel images of RNA RT-PCR products for amplicons of 236, 484, and 766 bp.

FIG. 6 are micrographs of rat liver tissue slides stained with S6 and NaKATPase reagents

### DETAILED DESCRIPTION

To more clearly and concisely describe and point out the subject matter of the claimed invention, the following definitions are provided for specific terms, which are used in the following description and the appended claims.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

As used herein, the term “biological sample” refers to a sample obtained from a biological subject, including sample of biological tissue or fluid origin obtained in vivo or in vitro. Such samples can be, but are not limited to, body fluid (e.g., blood, blood plasma, serum, or urine), organs, tissues, fractions, and cells isolated from mammals including, humans. Biological samples also may include sections of the biological sample including tissues (e.g., sectional portions of an organ or tissue). Biological samples may also include extracts from a biological sample, for example, an antigen from a biological fluid (e.g., blood or urine), or for example a nucleic acid extracted from biological tissue (e.g. breast, lung or prostate tissue) for nucleic acid sequencing. Biological samples may also include tissue portions cut from a paraffin block directly or indirectly or tissue section, such as a specific region of interest (ROI), that are digested and may be subject to analysis e.g. nucleic acid analysis by sequencing.

A biological sample may be of prokaryotic origin or eukaryotic origin (e.g., insects, protozoa, birds, fish, reptiles). In some embodiments, the biological sample is mammalian (e.g., rat, mouse, cow, dog, donkey, guinea pig, or rabbit). In certain embodiments, the biological sample is of primate origin (e.g., example, chimpanzee, or human).

As used herein “fixation” refers to a chemical process by which biological tissues are preserved from decay, thereby preventing autolysis or putrefaction. Fixation terminates any ongoing biochemical reactions, and may also increase the mechanical strength or stability of the treated tissues.

As used herein, the term “solid support” refers to an article on which targets present in the biological sample may be immobilized and subsequently detected by the methods disclosed herein. Targets may be immobilized on the solid support by physical adsorption, by covalent bond formation, or by combinations thereof. A solid support may include a polymeric, a glass, a paper such as FTA® paper, or a metallic material. Examples of solid supports include a membrane, a microtiter plate, a bead, a filter, a test strip, a slide, a cover slip, and a test tube.

As used herein, the term “target,” refers to the component of a biological sample that may be detected when present in the biological sample. The target may be any substance for which there exists a naturally occurring specific binder (e.g., an antibody), or for which a specific binder may be prepared (e.g., a small molecule binder or an aptamer). In general, a binder may bind to a target through one or more discrete chemical moieties of the target or a three-dimensional structural component of the target (e.g., 3D structures resulting from peptide folding). The target may include one or more of natural or modified peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, antigens, or haptens. In some embodiments, targets may include proteins or nucleic acids.

The disclosed methods relate generally to tissue fixation of a biological sample by contacting the sample with a reagent for fixation at a temperature of less than 20° C. In certain embodiments, the reagent comprises an aqueous buffer solution comprising 2-80 volume % of a water soluble alkylnitrile, C₂ to C₆ alkyl ester, or combination thereof and, 0.5 to 20% w/v formaldehyde to the aqueous buffer solution.

In certain preferred embodiments, the aqueous buffer solution comprises 10-50 volume % of a water soluble alkylnitrile, C₂ to C₆ alkyl ester, or combination thereof. In certain preferred embodiments, the formaldehyde to the aqueous buffer solution is present at 2-10% w/v and most preferably at approximately 4% w/v.

In certain embodiments, the water soluble alkylnitrile or C₂ to C₆ alkyl ester is acetonitrile, propionitrile, ethyl acetate, methyl acetate, methyl formate, or a combination thereof. In certain embodiments, the alkylnitrile is acetonitrile. In certain embodiments, the solution may further comprise other solvents such as alcohols.

The solution provides for complete fixation at colder temperature while maintaining morphological integrity and a corresponding increase in the quantity and quality of DNA and RNA preservation in the fixed tissues. In certain embodiments, the aqueous buffer solution comprise a buffer, such as but not limited to, a phosphate buffer to keep the pH of the aqueous solution between a pH of 4 to 8 and more preferable at a pH of approximately 7.

Tissue from biopsy or resection is typically preserved by treatment with 10% aqueous formalin (4% formaldehyde) at room temperature for morphological analysis, and, more recently, immunohistochemical analysis. Recent developments in next generation sequencing have made analysis of DNA and RNA from preserved tissue a top priority, but unfortunately standard formalin fixation compromises the quality and quantity of these analytes in fixed tissue. While it is known that fixation at colder temperatures can help preserve sensitive analytes, unfortunately cold fixation is much slower and typically results in underfixed tissue and poor tissue morphology. As such the addition of the acetonitrile provides added benefits.

In certain embodiments, the added solvent may not increase permeability of formaldehyde into tissue, rather it may increase the rate of reaction of formaldehyde with proteins and other materials that form the crosslinks that are critical for tissue fixation. As such the alkylnitrile or C₂ to C₆ alkyl ester may be responsible for making the solid tissue slightly more fluid, thus making it easier and faster for the solid state chemistry of formaldehyde crosslinking to occur, especially at lower temperature. Alternate chemistries, in particular formalin reaction with RNA and DNA nucleobases and nuclease degradation of DNA and RNA, may be hindered at lower temperatures. Thus increasing the rate of the desirable crosslinking, with added alkylnitrile or C₂ to C₆ alkyl ester at lower temperatures, while decreasing the negative reactions of DNA and RNA (also at lower temperatures) can yield a much more selective and effective fixation process.

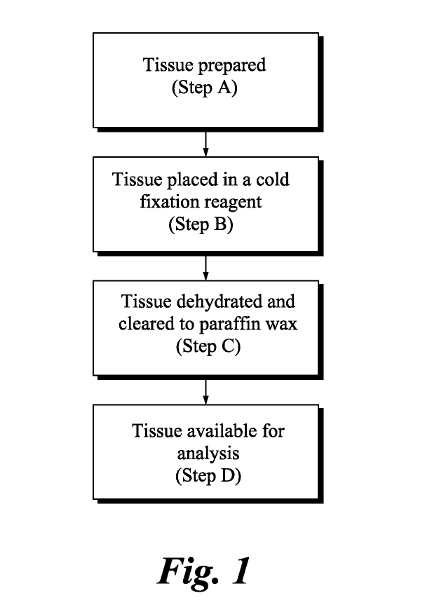

FIG. 1 is a schematic representation of one embodiment of the method wherein a biological sample, such as a tissue sample is prepared for fixation (Step A). The sample is placed in a cold fixation reagent below 20° C. In certain embodiments the fixation reagent comprises an aqueous buffer solution comprising 2-80 volume % of a water soluble alkylnitrile, C₂ to C₆ alkyl ester, or combination thereof and, 0.5 to 20% w/v formaldehyde to the aqueous buffer solution. (Step B) In certain embodiments the alkylnitrile is acetonitrile. In certain embodiments, the buffer solution may be a phosphate buffer. In certain preferred embodiments, the temperature of the solution is between 2° C. and 10° C., and more preferably between 2° C. and 5° C.

After a period of time and conditions, for example, 15 minutes to 24 hours, the tissue sample may undergo further processing. In certain embodiments, the tissue sample may be processed using standard protocols such as dehydration, clearing and immersing and embedding in paraffin wax (Step C).

For example in certain embodiments, standard protocols may involve removing the sample from the fixation reagent, washing the sample in a buffer solution (4° C., 1 hour), processing using a tissue processor for routine dehydration, clearing, and finally embedding in wax. The tissue may also be sectioned for analysis (Step D)

In still other embodiments, the method may further include the step of washing the biological sample with a rinsing liquid comprising water, a buffer solution or a combination after immersion in the fixation reagent in Step B. In certain preferred embodiments, the washing occurs at a temperature below 20° C. The washing may be by rinsing the sample or by immersion for a specific period of time.

In still other embodiments, a change in fixation temperature may occur to provide multiple stage cooling or heating. For example in one embodiment, the biological sample is contacted with the reagent at less than 20° C. for at least 1 hour and then heated to greater than 20° C. for an additional period of time.

In certain embodiments, the sample may also be subjected to a single analysis technique or a combination of techniques involving morphology with or without extraction methods. Analysis techniques may include, but are not limited to, DNA analysis or amplification, RNA analysis or amplification, nucleic acid sequencing, protein analysis, antigen retrieval, Hematoxylin and Eosin staining (H&E), immunofluorescence staining (IF), immunohistochemical staining (IHC), fluorescent in-situ hybridization (FISH), or other histological and morphological staining techniques.

As such, in certain embodiments, the sample may be subjected to extraction methods after fixation. The extraction methods include, but not are limited to, extraction of DNA, RNA, proteins, or analytes that provide additional information on the sample such as genetic, proteomic, or molecular profiling. For example the sample may be subjected to, DNA analysis or amplification, RNA analysis or amplification, nucleic acid sequencing, protein analysis, digestive treatment, or antigen retrieval.

In certain embodiments the biological sample may contain multiple targets adhered to a solid support In some embodiments, a biological sample may include a tissue sample, a whole cell, a cell constituent, a cytospin, or a cell smear. In some embodiments, a biological sample essentially includes a tissue sample or tissue components. A tissue sample may include a collection of similar cells obtained from a tissue of a biological subject that may have a similar function. In some embodiments, a tissue sample may include a collection of similar cells obtained from a tissue of a human. Suitable examples of human tissues include, but are not limited to, (1) epithelium; (2) the connective tissues, including blood vessels, bone and cartilage; (3) muscle tissue; and (4) nerve tissue. The source of the tissue sample may be solid tissue obtained from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject.

In certain embodiments the biological sample may be in suspension such as, but not limited to, a hematopoetic cell or circulating tumor cell in a biological fluid including a blood sample. As such in certain embodiments, detecting DNA, RNA, and protein targets including antigens, in a biological sample includes sequential detection of targets in the biological sample wherein the biological sample is in suspension; for example an in situ hybridization reaction in solution. In these instances, the biological sample must first be isolated from the suspension.

In some embodiments, the tissue sample may include primary or cultured cells, circulating disease or normal cells for example circulating tumor cells, activated leukocytes responding to an infectious agent, or cell lines.

In some embodiments, a biological sample includes tissue sections from healthy or diseased tissue samples (e.g., tissue section from colon, breast tissue, and prostate). A tissue section may include a single part or piece of a tissue sample, for example, a thin slice of tissue or cells cut from a tissue sample. In some embodiments, multiple sections of tissue samples may be taken, e.g. a tissue microarray, and subjected to analysis, provided the methods disclosed herein may be used for analysis of the same section of the tissue sample with respect to at least three different types of targets (at molecular level, e.g. an RNA, a protein and a DNA). In some embodiments, the same section of tissue sample may be analyzed with respect to at least four different targets (at morphological or molecular level). In some embodiments, the same section of tissue sample may be analyzed with respect to greater than four different targets (at morphological or molecular level). In some embodiments, the same section of tissue sample may be analyzed at both morphological and molecular levels.

In certain embodiments, a tissue section may undergo fixation and then further microtomed for analysis. In general a tissue section, if employed as a biological sample may have a thickness in a variety of ranges and sizes. For example in certain embodiments the tissue section may be fixed at approximately 3 mm and microtomed, after fixation and embedding, to approximately 5 micrometers. In certain embodiments, larger tissue samples may be used, in particular allowing for fixation of sample greater than 3 mm.

In some embodiments, a biological sample or the targets in the biological sample may be adhered to a solid support. A solid support may include microarrays (e.g., DNA or RNA microarrays), gels, blots, glass slides, beads, or ELISA plates. In some embodiments, a biological sample or the targets in the biological sample may be adhered to a membrane selected from nylon, nitrocellulose, and polyvinylidene difluoride. In some embodiments, the solid support may include a plastic surface selected from polystyrene, polycarbonate, and polypropylene. In certain embodiments, the solid support is glass.

In certain embodiments, the targets in the biological sample may include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, antigens, or haptens. In some embodiments, targets may essentially include proteins or nucleic acids. One or more of the aforementioned targets may be characteristic of particular cells, while other targets may be associated with a particular disease or condition. In some embodiments, targets that may be detected and analyzed using the methods disclosed herein may include, but are not limited to, prognostic targets, hormone or hormone receptor targets, lymphoid targets, tumor targets, cell cycle associated targets, neural tissue and tumor targets, or cluster differentiation targets

Suitable examples of prognostic targets may include enzymatic targets such as galactosyl transferase II, neuron specific enolase, proton ATPase-2, or acid phosphatase.

The detection of RNA generally involves an optional prehybridization step usually with salmon sperm DNA or tRNA for blocking followed by a hybridization step using sequence-specific probes to targets of interest at elevated temperature. In the absence of a prehybridization step, blocking agent is used with the probe itself during the hybridization step. Optimum probe concentration and temperature are generally empirically determined for best signal to noise ratio but are a function of probe Tm, buffer composition and probe type, e.g. LNA vs. DNA backbones. Hybridization time can also vary significant from about an half an hour or less to overnight hybridization and can be controlled by probe concentration. Post hybridization sample are subjected to one or more stringent washes to remove excess and non-specifically bound probe. Finally the probe is detected either directly if a signal generator is directly attached to the probe or indirectly with or without signal amplification. Detection may occur using a variety of techniques, including but not limited to manual observation, film or other recording devise, cameras, video recordings or a combination thereof. In some embodiments, the signal may be removed by the methods discussed above by chemical inactivation and sample may be probed for additional RNA species. Alternatively in other embodiments where the next step is protein detection, signal may be removed during the antigen retrieval step by denaturation of the bound probe or inactivation of signal due to antigen retrieval process that involves high temperature heating in acid and/or base.

In certain embodiments, the aforementioned biological sample may then be subjected to antigen retrieval and detection as for example, a method of protein detection. An antigen target may be present on the surface of a biological sample (for example, an antigen on a surface of a tissue section). In some embodiments, an antigen target may not be inherently present on the surface of a biological sample and the biological sample may have to be processed to make the target available on the surface (e.g., antigen recovery, enzymatic digestion or epitope retrieval).

In certain embodiments, the fixation conditions (FIG. 1, Step B) may further comprise exposure of the sample to multiple sequential temperatures, multiple sequential concentrations of fixation reagents, ultrasound acoustic energy, or a combination thereof.

### Experimental

### Selection Criteria

Traditional formalin fixations produce fragmented DNA and RNA and unwanted functionalization of the nucleobases, while non-formalin based fixations have negative effects on tissue morphology. One approach to avoiding nucleic acid damage without changing morphology would be to keep the basic formalin approach, but alter the formulation and fixation conditions. This would involve promoting the crosslinking of components such as the ε-amino group of lysine, while suppressing formaldehyde reaction with the nitrogens in the DNA and RNA nucleobases. Selectivity problems of this type are common in organic chemistry, and are typically solved by altering reagents and reaction conditions.

Running reactions at lower temperatures will often increase selectivity in organic chemistry, and low temperature may have the same effect on formalin fixation. Lower temperatures, especially when implemented immediately after tissue resection can also help inactivate nuclease activity, another well-known pathway for degradation of DNA and RNA. Unfortunately, simply running the traditional formalin fixation method at low temperature (4° C.) results in a reaction that is too slow to be of significant clinical value. Other recent efforts around faster cold fixation protocols include adding a robust dehydration step or a heated formalin step: “2+2” fixation.

To take advantage of any increased selectivity with cold fixation, a preliminary screen for co-solvents that might increase the kinetics of formalin fixation at low temperatures was undertaken. 1 cm³ Beef liver cubes were subjected to different fixation conditions, and the amount of fixation as well as any negative changes to tissue morphology was recorded. Formalin is known to fix tissue samples from the outside in, accompanied by a color change as the tissue becomes fixed, therefore the rate of fixation should correspond to the average thickness of the exterior fixed section of the tissue, which can easily be measured after slicing the tissue in half. Another parameters to assess is color. Color changes reflect morphology changes, and because we want the morphology of the cold-fixed tissue to be comparable to standard, room temperature fixed tissue, we will want the colors of the tissue sections to be comparable in cold-fixed tissue and standard fixed tissue. In addition, any tissue distortion or shrinkage will be noted, as this is highly undesirable in fixed tissue. While this screen was designed to look to identify co-solvents that increase the rate of formalin fixation at lower temperatures, the same cosolvents might also increase the rate of formalin fixation at room temperature and elevated temperature relative to the standard formalin formulation.

### Preliminary Screen

32 g of paraformaldehyde was heated with stirring in a capped flask with 640 ml of 100 mM pH 7.0 phosphate buffer at an oil bath temperature of 80° C. until all of the paraformaldehyde was dissolved, yielding a solution of 5% w/v formaldehyde in buffer. The mixture was then cooled in an ice bath to a temperature of approximately 10° C. 24 ml aliquots of this solution were then metered into vials, and 6 ml of co-solvent was added for fixative solutions that contained 20% v/v co-solvent. Co-solvents that were not soluble at 20% were added at 10% or 5% v/v as 3 ml aliquots with 3 ml of buffer or 1.5 ml aliquots with 4.5 ml buffer, respectively. Control samples with no co-solvent received 6 ml of additional buffer solution. The vials were capped, and stored overnight at 4° C. or 25° C. Fresh beef liver was procured and sliced into 1 cm³ cubes, avoiding the external liver membrane and internal large vasculature or connective tissue. Cubes were placed in the fixative vials, and then stored at the designated temperatures for 20 hours. At this time the liver samples were removed and visually inspected, then sliced in half and inspected once again. Samples sliced in half typically show a more “fixed” exterior section which is stiffer than the interior section, with different colors for each section and a reasonably sharp line separating the two sections. The average thickness of the exterior section was recorded for each, as well as the colors of the two sections. In addition, the distortion and or shrinkage of each tissue were graded on a scale of 0-4, with 0 being no distortion/shrinkage, and 4 being maximum distortion shrinkage. Samples with a score of zero have the smooth edges and shape of the original unfixed cube; samples with higher scores are highly puckered and shrunken. These results are shown in Table 1.

TABLE 1
Condition parameters used in sample screening.

| | | | | Exterior | | |
|---|---|---|---|---|---|---|
| | | | | Fixation | Distortion/ | |
| | | | Temp | Depth | Shrinkage | Exterior color/Interior |
| | Co-Solvent | Percent | (° C.) | (mm) | score | color |
| 1 | none | — | 25 | 3 | 0 | Tan/Light Pink |
| 2 | none | — | 4 | 1 | 0 | Tan/Red |
| 3 | Ethylene glycol | 20 | 4 | 1 | 4 | Red/Dark Red |
| 4 | Propylene glycol | 20 | 4 | 0 | 4 | All Dark Red |
| 5 | PEG350-monoMe | 20 | 4 | 1 | 4 | Dark Brown/Dark Red |
| 6 | acetonitrile | 20 | 4 | 3 | 0 | Tan/Light Pink |
| 7 | propionitrile | 10 | 4 | 2 | 1 | Tan/Light Pink |
| 8 | 1,4-dioxane | 20 | 4 | 2 | 2 | Brown/Red |
| 9 | tetrahydrofuran | 5 | 4 | 2 | 3 | Dark Brown/Dark Red |
| 10 | N-methylpyrrolidinone | 20 | 4 | 1 | 4 | Dark Brown/Dark Red |
| 11 | dimethyformamide | 20 | 4 | 1 | 3 | Dark Brown/Dark Red |
| 12 | Dimethylsulfoxide | 20 | 4 | 1 | 4 | Dark Brown/Dark Red |
| 13 | acetone | 20 | 4 | 2 | 3 | Dark Brown/Dark Red |
| 14 | Methyl formate | 5 | 4 | >5 | 1 | Greyish tan |
| 15 | 2-methoxyethanol | 20 | 4 | 1 | 2 | Brown/Red |
| 16 | Methyl acetate | 10 | 4 | >5 | 2 | Tan |
| 17 | Ethyl acetate | 5 | 4 | 5 | 1 | Tan/Light Pink |
| 18 | 1,3-butanediol | 20 | 4 | 1 | 4 | Dark Brown/Dark Red |
| 19 | Ethylene glycol | 20 | 4 | 1 | 3 | Tan/Pink/Dark Red (3 |
| | dimethyether | | | | | different consecutive |
| | | | | | | sections) |
| 20 | dimethylacetamide | 20 | 4 | 1 | 3 | Dark Brown/Dark Red |

From the screen, it is clear that cold fixation with 4% formaldehyde in buffer is slower than room temperature fixation with the same reagents (example 1 vs. example 2). There are a few co-solvents that appear to speed up cold fixation without significant tissue shrinkage, including acetonitrile, propionitrile, methyl formate, ethyl formate, and ethyl acetate. Most of the tested co-solvents have minimal effect on the amount of fixation, based on their exterior fixation depth measurement, and also have a negative effect on tissue quality, with significant tissue distortion and/or tissue shrinkage. Acetonitrile, with no observable distortion or shrinkage and a significant improvement in cold fixation rate appears to be the best of the co-solvents tested in this experiment for cold fixation, and may also have value in improving fixation results at other temperatures. Propionitrile, ethyl acetate, methyl acetate and methyl formate also speed up fixation with minimal distortion/shrinkage, and would also be valuable fixation co-solvents.

### Preparation of Solutions for Fixation of Rat Tissue Samples

4% Formaldehyde in 20% Acetonitrile/80% 100 mM pH 7.0 phosphate buffer: 32 g of paraformaldehyde was heated with stirring in a capped flask with 640 ml of 100 mM pH 7.0 phosphate buffer at an oil bath temperature of 80° C. until all of the paraformaldehyde was dissolved, yielding a solution of 5% w/v formaldehyde in buffer. The mixture was then cooled in an ice bath to a temperature of approximately 10° C., and 160 ml of acetonitrile was added. The solution was capped and stored overnight at 4° C. before use. 100 mL aliquots were used for tissue fixations.

4% Formaldehyde in 50% Acetonitrile/50% 20 mM pH 7.0 phosphate buffer: 32 g of paraformaldehyde was heated with stirring in a capped flask with 400 ml of 20 mM pH 7.0 phosphate buffer at an oil bath temperature of 80° C. until all of the paraformaldehyde was dissolved, yielding a solution of 8% w/v formaldehyde in buffer. The mixture was then cooled in an ice bath to a temperature of approximately 10° C., and 400 ml of acetonitrile was added. The solution was capped and stored overnight at 4° C. before use. The lower buffer concentration was required to avoid precipitation of buffer salts during tissue fixation. 100 mL aliquots were used for tissue fixations. 4% Formaldehyde in pH 7.0 phosphate buffer was purchased from Fisher Scientific.

### Fixations of Rat Tissue

Fresh rat tissues were collected according to an approved Animal Care and Use Protocol (ACUP). Tissues experienced cold ischemia conditions for less than 5 minutes prior to being placed in fixative. The median lobe of liver was excised from carcass, cut into 3 mm pieces and each piece was placed into a tissue cassette. When required, colon and muscle sections were also excised and cut into 3 mm sections and placed in cassettes. The tissue cassette was submersed in fixative (approximately 100 mL) pre-equilibrated to proper temperature for the desired duration of time at the desired temperature (see Table 1). For ultrasound assisted fixations, the tissue cassette was placed in the designated fixative bath pre-equilibrated to the desired temperature in a Jokoh Histra DC ultrasound unit, and exposed to ultrasonic irradiation for 30 minutes with heating or cooling as required to maintain temperature. For fixations run at multiple temperatures, cassettes were first placed in fixative at the initial temperature for 2 hours, and then transferred to the proper fixative solution at the second temperature for an additional 2 hours. After the fixation incubation, the tissue cassette was placed in an aqueous wash solution of either PBS or 100 mM pH 7.0 phosphate buffer for one hour at 4° C., or placed directly into the retort of the tissue processor. If a wash was conducted, tissue was placed into the tissue processor retort immediately following the wash step. Experiments were designed so that all fixations with or without post-fixation treatment finished at the same time so that none of the samples had to wait for more than a few minutes between fixation/post-fixation treatment and the start of tissue processing.

The tissue processor (Sakura, Tissue-Tek, VIP6) began immediately after all tissue cassettes were loaded into the retort. The processor steps are outlined in Table 2 below.

TABLE 2
Processor steps employed.

| Step | Reagent | Duration | Temperature | Pressure/Vacuum |
|---|---|---|---|---|
| 1 | 70% Ethanol | 40 min | 37° C. | Yes |
| 2 | 80% Ethanol | 40 min | 37° C. | Yes |
| 3 | 95% Ethanol | 40 min | 37° C. | Yes |
| 4 | 95% Ethanol | 40 min | 37° C. | Yes |
| 5 | 100% Ethanol | 40 min | 37° C. | Yes |
| 6 | 100% Ethanol | 40 min | 37° C. | Yes |
| 7 | Xylene | 40 min | 37° C. | Yes |
| 8 | Xylene | 40 min | 37° C. | Yes |
| 9 | Paraffin | 40 min | 56° C. | Yes |
| 10 | Paraffin | 40 min | 56° C. | Yes |
| 11 | Paraffin | 40 min | 56° C. | Yes |
| 12 | Paraffin | 40 min | 56° C. | Yes |

Tissues were in liquid paraffin wax (56° C.) until removed from processor; removal was within 10 minutes of processing completion. Tissues were embedded in paraffin blocks and stored at 4° C. until sectioned. Sectioning was conducted on a Leica microtome (model # RM2265) at room temperature. Each tissue block was faced, where excess wax was cut away until the tissue is exposed, and then an additional 750 um was cut (to get into the tissue rather than the surface). Tissue was sectioned at Sum thick and floated in a nuclease free water bath set at 50° C. to flatten the tissue. The float was momentary and the tissue was collected onto a poly-L-lysine coated glass microscope slide (Fisherbrand, Colorfrost Plus, cat #12-550-20). The tissue slide was dried at room temperature for approximately 3 hours then stored at 4° C. until analysis.

### Histological Analysis of Fixed Rat Tissue

H&E staining was completed manually according to the steps in Table 3 below (all at room temperature). Note, immediately prior to step 1 in Table 3, slides were warmed in an oven set to 60° C. for 15 minutes.

TABLE 3
H&E Staining Protocol

| Step | Solution | Time | |
|---|---|---|---|
| 1 | Xylene | 5 | min |
| 2 | Xylene | 3 | min |
| 3 | 100% Alcohol | 1 | min |
| 4 | 100% Alcohol | 1 | min |
| 5 | 95% Alcohol | 1 | min |
| 6 | 95% Alcohol | 1 | min |
| 7 | 80% Alcohol | 1 | min |
| 8 | DI Water | 20-30 | sec |
| 9 | Hematoxylin | 5 | min |
| 10 | DI Water | 5 | quick dips |
| 11 | DI Water | 5 | quick dips |
| 12 | 1% Acid alcohol | 1 | quick dip |
| 13 | DI Water | 5 | quick dips |
| 14 | DI Water | 5 | quick dips |
| 15 | Blueing | 2 | min |
| 16 | Running water | 2 | min |
| 17 | DI water | 5 | quick dips |
| 18 | 95% Alcohol | 30 | sec |
| 19 | Eosin Y | 90 | sec |
| 20 | 95% Alcohol | 10 | dips |
| 21 | 95% Alcohol | 10 | dips |
| 22 | 100% Alcohol | 30 | sec |
| 23 | 100% Alcohol | 30 | sec |
| 24 | Xylene | 3 | min |
| 25 | Xylene | 3 | min |
| 26 | Coverslip | | |

After H&E staining, coverslips were affixed to the slides using permanent mounting media. Tissues were then viewed/imaged with a 20× objective on the Olympus VS120 microscope.

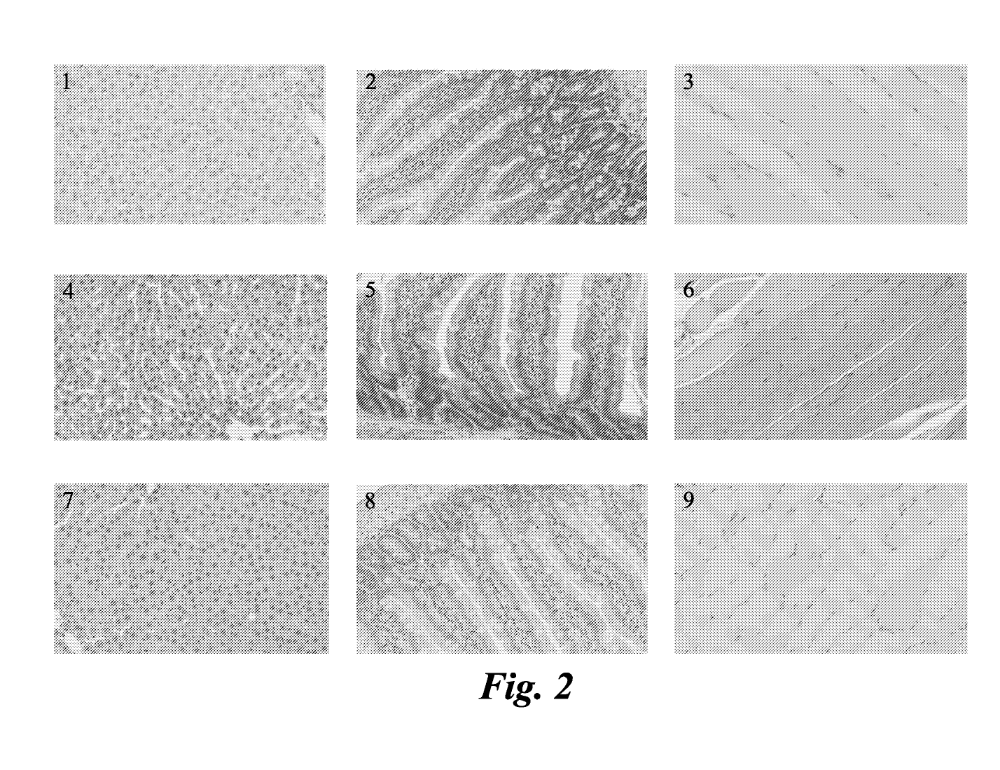

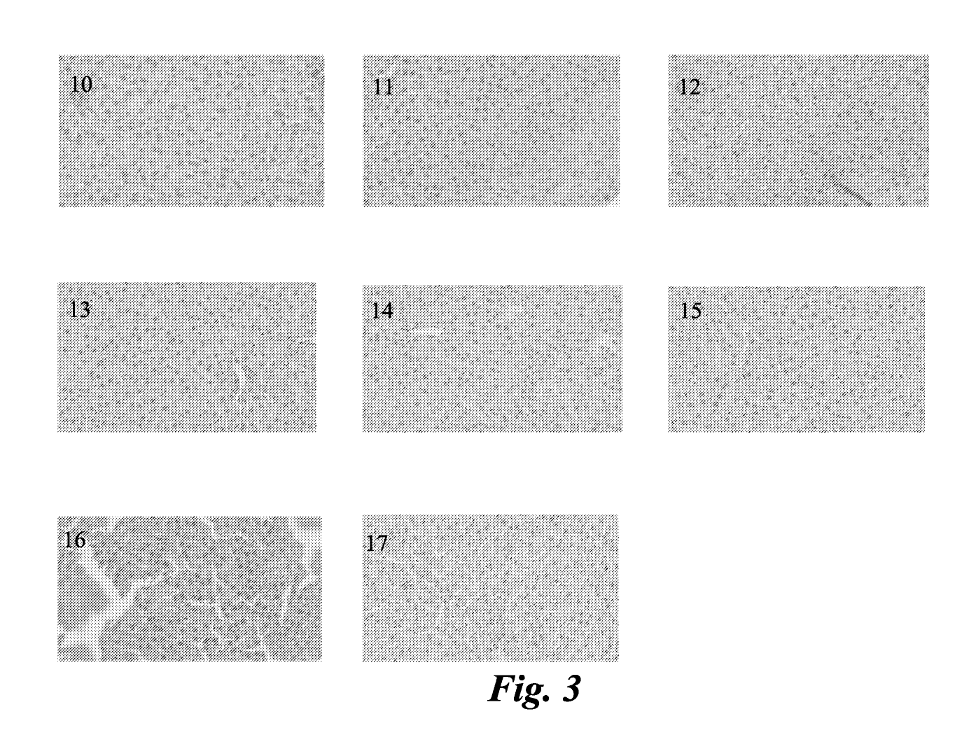

Table 4 lists various fixation processes at various conditions, including different fixation solvents, temperature, time and with and without the use of ultrasound assisted fixation for liver, colon, and muscle tissues. Following fixation each sample was processed following the steps in Table 2, and H&E stained following the steps in Table 3. FIG. 2 and FIG. 3 show micrographs of H&E treated tissues obtained using a 20× objective lens for each of the conditions listed in Table 4. 1 through 9 in FIG. 2 compare standard room temperature formalin fixation for rat liver, colon, and muscle (No. 1-3, respectively) and low temperature aqueous formalin fixation (No. 4-6 respectively). The low temperature aqueous fixation is clearly underfixed, note the gaps and cracking in Nos. 4-6. In an embodiment of the invention, acetonitrile added to the aqueous fixation solution at low temperature (No. 7-9) yields fixed tissues with H&E results comparable to tissue from standard room temperature all-aqueous formalin fixation. As shown, the preservation of native structure is improved with the use of acetonitrile in the fixation medium at low temperature.

TABLE 4
H&E fixative reagents and conditions.

| | | | Time | Temp | |
|---|---|---|---|---|---|
| No. | Fixative | Tissue | (hr) | (° C.) | Additional comments |
| 1 | 4% Formaldehyde in phosphate buffer | Liver | 24 | 25 | |
| 2 | 4% Formaldehyde in phosphate buffer | Colon | 24 | 25 | |
| 3 | 4% Formaldehyde in phosphate buffer | Muscle | 24 | 25 | |
| 4 | 4% Formaldehyde in phosphate buffer | Liver | 24 | 4 | |
| 5 | 4% Formaldehyde in phosphate buffer | Colon | 24 | 4 | |
| 6 | 4% Formaldehyde in phosphate buffer | Muscle | 24 | 4 | |
| 7 | 4% Formaldehyde in 80:20 phosphate buffer:CH₃CN | Liver | 24 | 4 | |
| 8 | 4% Formaldehyde in 80:20 phosphate buffer:CH₃CN | Colon | 24 | 4 | |
| 9 | 4% Formaldehyde in 80:20 phosphate buffer:CH₃CN | Muscle | 24 | 4 | |
| 10 | 4% Formaldehyde in 80:20 phosphate buffer:CH₃CN | Liver | 0.5 | 25 | Ultrasound Assisted |
| | | | | | Fixation |
| 11 | 4% Formaldehyde in phosphate buffer | Liver | 0.5 | 50 | Ultrasound Assisted |
| | | | | | Fixation |
| 12 | 4% Formaldehyde in 80:20 phosphate buffer:CH₃CN | Liver | 23 | 4 | 1 hr 4° C. PBS wash |
| | | | | | post-fixation |
| 13 | 4% Formaldehyde in 80:20 phosphate buffer:CH₃CN | Liver | 23 | 4 | 1 hr 4° C. 100 mM |
| | | | | | phosphate buffer |
| | | | | | wash post-fixation |
| 14 | 4% Formaldehyde in 50:50 phosphate buffer:CH₃CN | Liver | 4 | 4 | |
| 15 | 4% Formaldehyde in 80:20 phosphate buffer:CH₃CN | Liver | 4 | 4 | 2 hr at 4° C. followed by |
| | | | | | 2 hr at 22° C. |
| 16 | 4% Formaldehyde in phosphate buffer | Liver | 4 | 4 | 2 hr at 4° C. followed by |
| | | | | | 2 hr at 22° C. |
| 17 | 4% Formaldehyde in 80:20 phosphate buffer:CH₃CN | Liver | 4 | 4 | 2 hr at 4° C. followed by |
| | | | | | 2 hr at 22° C. in 4% |
| | | | | | Form in 50:50 |
| | | | | | buffer:CH₃CN |

Recovery of Amplifiable DNA from Fixed Rat Liver Tissue

The percentage of intact DNA was determined for tissue slides with several of the different fixation conditions in Table 4 for 4 different amplicons.

5 μm fixed tissue sections were deparaffinized in glass coplin jars by two successive 5 minute washes in HistoChoice®. The tissue was then rehydrated in 5 minute washes of 95%, 70%, and 50% ethanol sequentially. Tissue sections were allowed to dry in the hood before removal from the slide, which was done by applying FFPE Digest Buffer (50 mM Tris pH7.4, 10 mM EDTA, 0.5% Sarkosyl, 50 mM NaCl, 1M NaSCN, and 0.5 mg/ml Proteinase K) and scraping the tissue section into a 200 μl reaction tube. Samples were then incubated at 50° C. for 2 hours or until the tissue was digested into a slurry. The samples were then applied to FTA® Classic paper and dried in a desiccator. Each DNA sample was then repaired with a repair reaction mix consisting of 2 μl NEBuffer 2, 2 μl 1 mM dNTP mix, 0.5 μl 10 mM ATP, 2 μl 1 mg/ml BSA, 10 μl 10% α-cyclodetrin, 0.66 μl 400 U/μl T4 DNA Ligase, 0.66 μl 10 U/μl Endonuclease IV, 0.66 μl 10 U/μl DNA Polymerase I, and 1.5 μl water. Repairs were performed directly with three 1.2 mm punches of FTA® paper containing the digested tissue. The reaction was incubated at 37° C. for one hour, then 85° C. for fifteen minutes. The DNA eluted from FTA® into the repair reaction solution was then quantified using the PicoGreen® assay. Using the concentration values from the PicoGreen® assay, 10 nanograms of FFPE DNA were added to qPCR reactions and the total amount of amplifiable DNA was determined by comparison to a standard curve of genomic DNA. The quantity of amplifiable DNA was calculated at several different amplicons of increasing size. Results were reported as a percentage of the 10 nanograms of DNA in the reactions for each amplicon individually, calculating actual amplifiable DNA amount using the resulting Ct values as they occur along a four log dilution standard curve of rat genomic DNA of known copy number in the relevant range.

The primer sequences used in the DNA integrity assay are as follows. Master Forward—GTAGTGGCTTAGTCCCTG (SEQ ID NO: 1), 90 base pair amplicon reverse—GAGAAAGAACTGGAAGAGC (SEQ ID NO: 2), 260 base pair amplicon reverse—CCCATACATATACAGCCAC (SEQ ID NO: 3), 370 base pair amplicon reverse—CACTCCTTCTCTAAAAGGG (SEQ ID NO: 4), 540 base pair amplicon reverse—GCAAATGGTTGGAACTGG (SEQ ID NO: 5), 829 base pair amplicon reverse—CTGGTACAACCATTCTGG (SEQ ID NO: 6), 1.2 kilobase pair amplicon reverse—GTAAGGCTAAGGACACC (SEQ ID NO: 7).

The qPCR reaction mix consists of 2.5 μl 10× AmpliTaq Gold Buffer, 2.5 μl 25 mM MgCl2, 0.5 μl 10 mM dNTP mix, 1 μl 12.5 μM Primer pair, 0.08 μl SYBR® Green dye, 0.05 μl ROX Dye, 0.13 μl AmpliTaq Gold DNA Polymerase, 2 μl of 5 ng/μl template, and 16.24 μl Water. The thermocycler program was 95° C. 10 min (Taq activation) followed by 40 repetitions of: 95° C. 15 s; 57° C. 30 s; 72° C. 60 s.

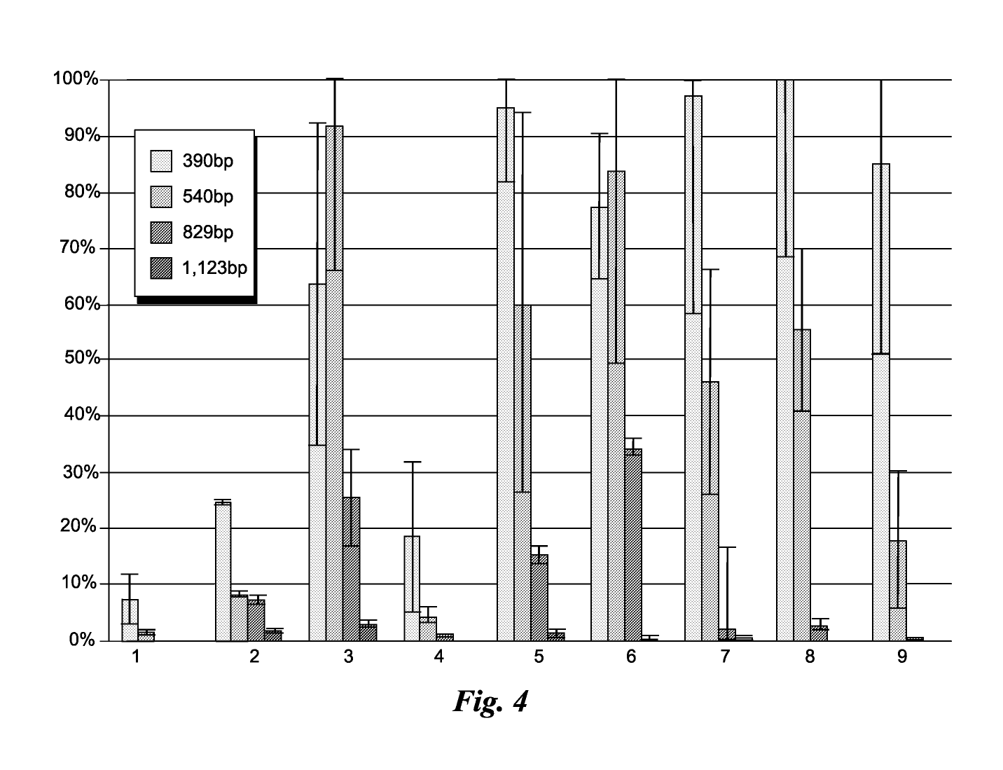

The increase in amplifiable DNA recovery under specific fixation conditions compared to standard aqueous room temperature fixation is apparent from the data summarized in Table 5 and FIG. 4. Increased amounts of amplifiable DNA are recovered for all amplicons (380, 540, 829, and 1123 base pairs) with rat liver tissue slides from samples fixed cold with 4% formaldehyde in 20% acetonitrile versus the standard room temperature fixation of 4% formaldehyde in aqueous buffer, compare entries 1 and 7 in Table 5. Note once again that both of these samples have comparable morphology, exemplified by micrographs 1 and 7 in FIG. 2. There is an additional improvement in recovery of amplifiable DNA from the cold 4% formaldehyde in 20% acetonitrile fixed tissue when the tissue sample is washed with an aqueous buffer solution for 1 hour immediately after tissue fixation and immediately before further tissue processing, compare 12 and 13 vs. 7 in Table 5 and FIG. 4. Note also samples 7, 12, and 13 are fully fixed based on their micrographs in FIGS. 2 and 3.

Higher concentrations of acetonitrile in the fixative can lead to shorter fixation times, as sample 14, from a cold formalin fixation with 50% acetonitrile after only 4 hours, is fully fixed based on the H&E data in FIG. 3. This sample also shows a significant improvement in amplifiable DNA recovery compared to the standard aqueous room temperature fixation. Enhanced recovery of amplifiable DNA is also possible with an initial formalin fixation at low temperature with added acetonitrile for a specified time followed by warming the sample and finishing the fixation at higher temperature, thus potentially allowing for a faster fixation than what is achievable if the fixation occurs only at cold temperature. This is exemplified in sample 15. Note that the 4 hour fixation in this case yields a fully fixed sample based on the H&E results in FIG. 3, with enhanced recovery of amplifiable DNA, exemplified in Table 5 and FIG. 4. Compare this to Fixation 16 in FIG. 3, a comparable time and temperature profile without added acetonitrile results in an underfixed sample. The same multiple temperature profile can also be used with a higher concentration of acetonitrile, note fixation 17, with excellent morphology (FIG. 3) and excellent recovery of amplifiable DNA (Table 5 and FIG. 4).

Added acetonitrile can also be used to lower the temperature in ultrasound assisted formalin fixations, resulting in an improvement in amplifiable DNA recovery. Formaldehyde fixation for 30 minutes at 25° C. with 20% acetonitrile and ultrasound irradiation results in fully fixed tissue (Fixation 10 in FIG. 3), while ultrasound assisted fixation with a comparable time and temperature profile but with an aqueous formulation led to underfixed tissue. The fixation had to be run at 50° C. in aqueous formaldehyde to achieve full fixation (Fixation 11 in FIG. 3), but this resulted in much lower yields of amplifiable DNA compared to the colder ultrasound assisted formalin fixation with added acetonitrile (Fixation 10 and 11 in Table 5 and FIG. 4).

TABLE 5
Percent Intact DNA under Various Process Conditions

| No. | Coditions | 380 bp | 540 bp | 829 bp | 1,123 bp |
|---|---|---|---|---|---|
| 1 | 4% Form in | 7.14% | 1.26% | 0.00% | 0.00% |
| | buffer 25° C. | | | | |
| 7 | 4% Form in 8:2 | 24.52% | 7.95% | 7.11% | 1.59% |
| | buffer:CH₃CN | | | | |
| | 4° C. | | | | |
| 10 | 4% Form in 8:2 | 63.42% | 91.28% | 25.41% | 2.72% |
| | buffer:CH₃CN | | | | |
| | Ultrasound 25° C. | | | | |
| 11 | 4% Form in | 18.18% | 4.09% | 0.54% | 0.00% |
| | buffer Ultrasound | | | | |
| | 50° C. | | | | |
| 12 | 4% Form in 8:2 | 95.1% | 60.2% | 15.1% | 1.1% |
| | buffer:CH₃CN | | | | |
| | PBS wash 4° C. | | | | |
| 13 | 4% Form in 8:2 | 77.3% | 83.3% | 34.3% | 0.0% |
| | buffer:CH₃CN | | | | |
| | buffer wash 4° C. | | | | |
| 14 | 4% Form in 1:1 | 97.1% | 45.9% | 1.8% | 0.3% |
| | buffer:CH₃CN | | | | |
| | 4° C. | | | | |
| 15 | 4% Form in 8:2 | 104.0% | 55.1% | 2.7% | 0.0% |
| | buffer:CH₃CN | | | | |
| | 2 hr at 4° C., | | | | |
| | 2 hr at 22° C. | | | | |
| 17 | 4% Form in 8:2 | 85.0% | 17.8% | 0.3% | 0.0% |
| | buffer:CH₃CN | | | | |
| | 2 hr at 4° C., 4% | | | | |
| | Form in 1:1 | | | | |
| | buffer:CH₃CN | | | | |
| | 2 hr at 22° C. | | | | |

RNA Recovery from Fixed Rat Liver Tissue

RNA was extracted from 5 nm tissue sections using the RNeasy FFPE Kit (Qiagen #73504) as per manufacturer's instructions. RNA was subjected to reverser transcription reaction using First Strand cDNA Synthesis kit (GE Healthcare #27-9261-01) as per manufacturer's instructions. RNA quality was determined by successful amplification of target amplicons of increasing size. The housekeeping gene beta-actin was used as screening PCR target. PCR results were visualized using 2% agarose gels stained with SYBR® Gold DNA stain.

The primer sequences used in the DNA integrity assay are as follows. Master Forward—GTAGTGGCTTAGTCCCTG (SEQ ID NO: 1), 90 base pair amplicon reverse—GAGAAAGAACTGGAAGAGC (SEQ ID NO: 2), 260 base pair amplicon reverse—CCCATACATATACAGCCAC (SEQ ID NO: 3), 370 base pair amplicon reverse—CACTCCTTCTCTAAAAGGG (SEQ ID NO: 4), 540 base pair amplicon reverse—GCAAATGGTTGGAACTGG (SEQ ID NO: 5), 829 base pair amplicon reverse—CTGGTACAACCATTCTGG (SEQ ID NO: 6), 1.2 kilobase pair amplicon reverse—GTAAGGCTAAGGACACC (SEQ ID NO: 7).

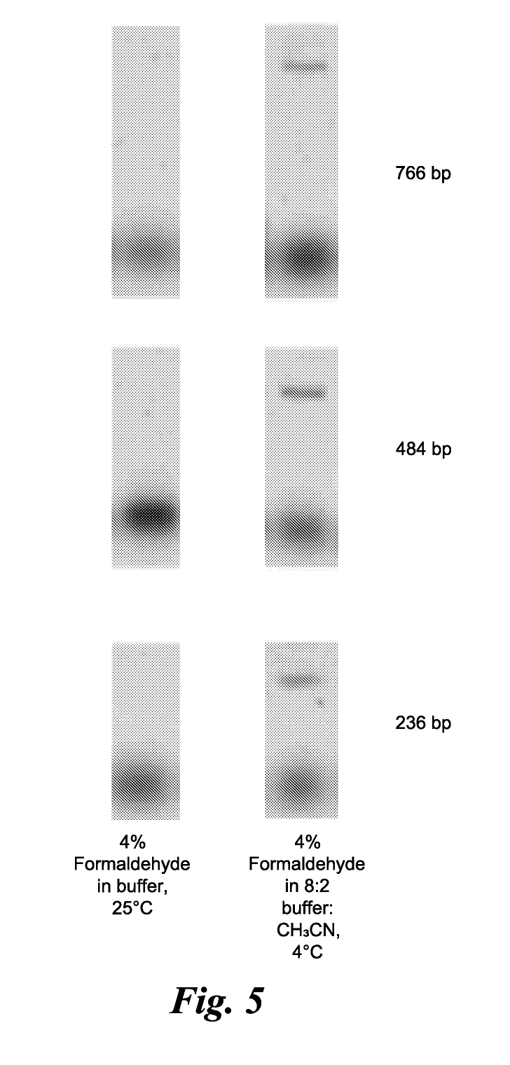

RNA amplification was similarly measured (236,484, and 766 bp) with an improved recovery from tissue slices fixed with 4% formaldehyde in 8:2 buffer:acetonitrile solution at 4° C. compared to tissue slices fixed with formaldehyde and buffer at 25° C. This is shown in FIG. 5, an image of RNA gels derived from tissue slices fixed by both methods. The improved RNA recovery with cold fixation in aqueous acetonitrile relative to standard aqueous fixation may be due to lower nuclease activity at lower temperatures, lower amount of reaction between formaldehyde and RNA at low temperatures, or both. The results in FIGS. 4 and 5 indicate that both DNA and RNA recovery can be dramatically improved with cold fixation with added acetonitrile, and the results in FIGS. 2 and 3 indicate that, while morphology suffers with fixed tissue prepared with cold, aqueous formaldehyde fixation, adding acetonitrile to the cold fixation formulation improves morphology so that H&E results are comparable to those from standard, room temperature aqueous fixations. The end result is a viable fixation method that dramatically improves the recovery of DNA and RNA from fixed tissue. The effort to increase recovery of DNA and RNA is evidence that the process most likely will increase recovery of other sensitive analytes, for example phosphoproteins.

### Immunofluorescence Staining Protocol

Immunofluorescence staining with S6 and NaKATPase help to visualize cytoplasm and cellular membranes, respectively. They offer a complimentary assessment of tissue morphology to standard H&E staining.

Slide baking was performed using a standard oven (Fisher Scientific), dewax and clearing were carried out by a Leica XL automated platform using the standard protocols as summarized in Table 2. ( ) Antigen retrieval was performed using a BioCare NxGen decloaker (SN 0243) and standard ARS1 (pH8) and ARS2 (pH6) reagents, according to the methods previously described (U.S. Pat. No. 8,067,241). Succeeding slide handling steps included post antigen retrieval washes, slide blocking and DAPI staining. Antibody staining was carried out using 150 μL of Ab solution at 1× the standard working concentration (see Table 3 for Ab concentrations) with direct conjugates for S6 and NaKAtpase targets. Antibody dead volumes were 50 μL/slide. All imaging steps were performed using an Olympus IX81 inverted fluorescence microscopy platform, supported with Image_app acquisition software. All slides were imaged using standard antifade mounting media. The manual staining platform utilized a stock concentration of 300 ug/ml and a working concentration of 5 ug/ml for the S6 marker and a stock concentration of 350 ug/ml and a working concentration of 5 ug/ml for the NaKATPase marker.

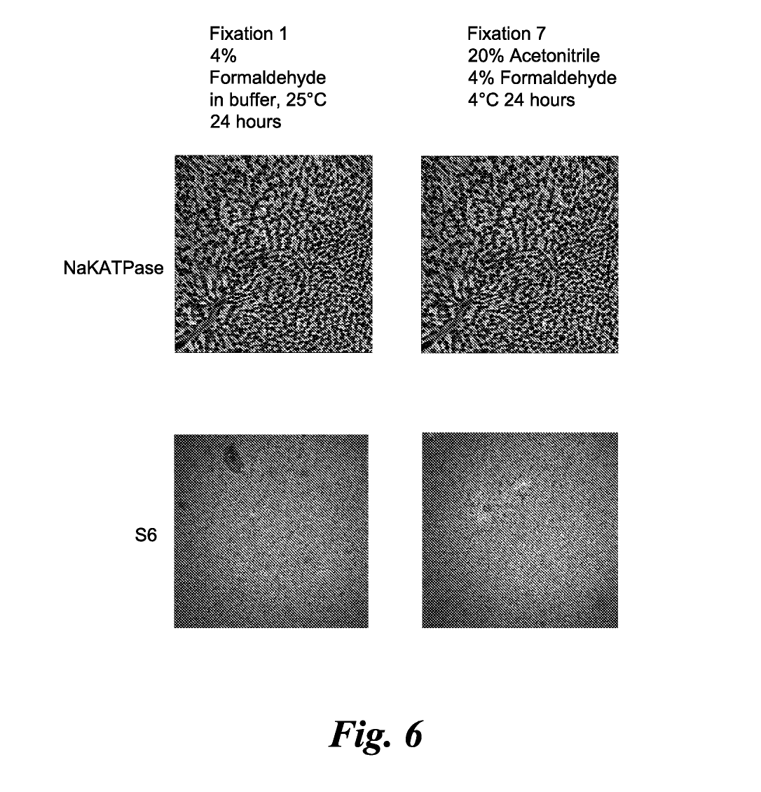

Rat liver tissues fixed under standard, room temperature 24 hour aqueous fixation conditions and at 4° C. with 20% acetonitrile for 24 hours have comparable S6 and NaKATPase staining patterns as shown in FIG. 6, validating the H&E data shown in FIGS. 2 and 3.

TABLE 6
Immunofluorescence Protocol summary.

| Protocol step | Summary |
|---|---|
| 1 | Bake and Dewax |
| 2 | Antigen retrieval using BioCare NxGen |
| | Decloaker |
| 3 | Block, and DAPI stain |
| 4. | Antifluorescence Imaging |
| 5 | S6 and NaKAtpase stain |
| 6 | Imaging |

TABLE 7
Immunofluorescence slide sample preparation (pre-staining steps).

| Step | Comment |
|---|---|
| Bake | 1 hr, 60° C. |
| Dewax | Xylene 5 min/2X |
| | 100% ETOH 5 min/2X |
| | 95% ETOH 5 min/2X |
| | 70% ETOH 5 min/2X |
| | 50% ETOH 5 min/2X |
| | PBS 5 min/2X |
| | 0.3% Triton X-100 + PBS 10 min |
| | PBS 5 min |
| Antigen | ARS1 20 min/ARS2 30 min |
| Retrival | |
| Block | 15 min |
| DAPI | 15 min |

The invention includes embodiments that relate generally to methods applicable in analytical, diagnostic, or prognostic applications such as analyte detection, histochemistry, immunohistochemistry, immunofluorescence, chromogenic in situ hybridization, or fluorescence in situ hybridization (FISH), nucleic acid sequencing, mass spectroscopy, optical spectrosopy. In some embodiments, the methods disclosed herein may be particularly applicable in histochemistry, immunostaining, immunohistochemistry, immunoassays, or immunofluorescence. In some embodiments, the methods disclosed herein may be particularly applicable in immunoblotting techniques, for example, western blots or immunoassays such as enzyme-linked immunosorbent assays (ELISA).

The invention may be embodied in other specific forms without departing from the spirit or essential characteristics thereof. The foregoing embodiments are therefore to be considered in all respects as illustrative rather than limiting on the invention described herein. The scope of the invention is thus indicated by the appended claims rather than by the foregoing description, and all changes that come within the meaning and range of equivalency of the claims are therefore intended to be embraced therein. The invention may be embodied in other specific forms without departing from the spirit or essential characteristics thereof. The foregoing embodiments are therefore to be considered in all respects as illustrative rather than limiting on the invention described herein. The scope of the invention is thus indicated by the appended claims rather than by the foregoing description, and all changes that come within the meaning and range of equivalency of the claims are therefore intended to be embraced therein.

1. A method of cold fixation of a biological sample comprising the steps of:
a. contacting the biological sample with a reagent for fixation at a temperature of less than 20° C., the reagent comprising:
an aqueous buffer solution comprising 2-80 volume % of a water soluble alkylnitrile, C₂ to C₆ alkyl ester, or combination thereof; and
0.5 to 20% w/v formaldehyde to the aqueous buffer solution; and
b. removing the biological sample from contact with the reagent.

2. The method of claim 1 where the buffer comprises organic or inorganic salts that maintain a pH of 4.0 to 8.0.

3. The method of claim 1 where the aqueous buffer solution comprising 10-50 volume % of a water soluble alkylnitrile, C₂ to C₆ alkyl ester, or combination thereof.

4. The method of claim 1 comprising 2-10% w/v formaldehyde to the aqueous buffer solution.

5. The method of claim 1 where the water soluble alkylnitrile and C₂ to C₆ alkyl ester comprises acetonitrile, propionitrile, ethyl acetate, methyl acetate, methyl formate, or a combination thereof.

6. The method of claim 5 where the alkylnitrile is acetonitrile.

7. The method of claim 1 where the contacting the biological sample with a reagent occurs at a temperature of less than 10° C.

8. The method of claim 7 where the temperature is between approximately 5° C. and 2° C.

9. The method of claim 1 further comprising washing the biological sample with a rinsing liquid comprising water, a buffer solution or a combination thereof.

10. The method of claim 9 wherein washing the biological with a rinsing liquid occurs at a temperature of less than 20° C.

11. The method of claim 1 wherein the biological sample is contacted with the reagent at less than 20° C. for at least one hour and then heated to greater than 20° C. for an additional period of time.

12. The method of claim 1 further comprises the step of applying ultrasound acoustic energy between steps a and b.

13. The method of claim 1 wherein the method further comprises dehydrating the sample, clearing, immersing and embedding the sample in paraffin wax.

14. The method of claim 13 wherein the sample undergoes DNA analysis or amplification, RNA analysis or amplification, nucleic acid sequencing, protein analysis, antigen retrieval, Hematoxylin and Eosin staining (H&E), immunofluorescence staining (IF), immunohistochemical staining (IHC), fluorescent in-situ hybridization (FISH), other histological and morphological staining techniques, or a combination thereof.

15. The method of claim 14 wherein the sample undergoes DNA analysis or amplification, RNA analysis or amplification or nucleic acid sequencing.

16. A reagent for fixation of a biological sample, the reagent comprising:
an aqueous buffer solution comprising 2-80 volume % of a water soluble alkylnitrile, C₂ to C₆ alkyl ester, or combination thereof; and
0.5 to 20% w/v formaldehyde to the aqueous buffer solution.

17. The reagent of claim 16 where the buffer comprises organic or inorganic salts that maintain a pH of 4.0 to 8.0.

18. The reagent of claim 16 where the water soluble alkylnitrile and C₂ to C₆ alkyl ester comprises acetonitrile, propionitrile, ethyl acetate, methyl acetate, methyl formate, or a combination thereof.

19. The reagent of claim 18 where the alkylnitrile is acetonitrile.

20. The reagent of claim 16 further comprising an alcohol.

21. A method of analyzing a biological sample comprising the steps of:
a. contacting the biological sample with a reagent for fixation at a temperature of less than 20° C., the reagent comprising;
an aqueous buffer solution comprising 2-80 volume % of a water soluble alkylnitrile, C₂ to C₆ alkyl ester, or combination thereof; and
0.5 to 20% w/v formaldehyde to the aqueous buffer solution;
b. removing the biological sample from contact with the reagent;
c. washing the biological sample with a rinsing liquid comprising water, a buffer solution or a combination thereof;
d. embedding the sample in paraffin wax through at least one of, dehydrating the sample, clearing the sample, and immersing and embedding the sample in paraffin wax;
e. analyzing the morphology of the sample; and
f. extracting an analyte of the sample for analysis wherein the analyte is a DNA, RNA, protein, or a combination thereof.
